(19) United States
(12) Patent Application Publication
(10) Pub. No.: US 2011/0105862 A1
(43) Pub. Date: May 5, 2011

(54) DEVICE FOR ACQUIRING AND PROCESSING PHYSIOLOGICAL DATA OF AN ANIMAL OR OF A HUMAN IN THE COURSE OF A PHYSICAL OR MENTAL ACTIVITY

(75) Inventors: Valentin Gies, Toulon (FR); Fabien Hasni, Plaisir (FR)

(73) Assignee: UNIVERSITE DU SUD TOULON-VAR, La Garde Cedex 20 (FR)

(21) Appl. No.: 12/990,062

(22) PCT Filed: Apr. 28, 2009
(86) PCT No.: PCT/FR2009/000495

§ 371 Date: Jan. 14, 2011

(30) Foreign Application Priority Data
Apr. 28, 2008 (FR) 08/02383

(51) Int. Cl. A61B 5/00

(52) U.S. Cl. 600/301

(57) ABSTRACT

The invention relates to a system allowing the acquisition and processing of data representative of the physical or mental activity and/or of the physiological state of human or animal individuals. This system includes, for each individual, a unique individual electronic box encasing several sensors capable of measuring physical and/or biological quantities related to the physical and/or biological activity of the wearer of the box and of returning information. Each electronic box moreover is provided with an interface for radio communication with a device allowing the management of the data gathered from the individual boxes and management.

The present invention relates to a device for acquiring and processing physiological data of an individual in the broad sense, whether an animal or a human being, in the course of a physical or mental activity.

The first application which this invention relates to is that of monitoring racehorses during training or racing, it being understood that other applications are envisaged in the field of the present invention with certain adaptations and variants of the basic data acquisition and processing device.

Another application sought is that of monitoring the physical performance of sportspersons when individual or collective sports are being practiced.

Another application sought, still without any limitative character, is that of monitoring physical, physiological and biological parameters of individuals subjected to psychological pressure or a stress, in particular of a work-related nature.

The invention will therefore be described mainly in the context of the application thereof to the monitoring of the training of racehorses, but without this application having any limitative character.

### STATE OF THE ART

In the field of training and monitoring racehorses, a device for the automatic training of horses is known from WO 01/97606. The aim pursued by this device is mainly enabling horses to be trained without human intervention. For this purpose, each horse is placed individually in an automated training installation, in which it can run either on a moving belt, or in a partitioned space that itself moves on a rail positioned around a racecourse, or in a circular training apparatus also referred as a “walker”. In order to monitor the performance of the horse during its training, electrodes are placed on the horse in order to collect various electrical signals corresponding the physiological activity of the horse, in particular the activity of the heart, the lungs and the muscles.

It is clear that this automated device does not correspond to the training of a horse under actual conditions on a racecourse and in the presence of a jockey, and has the sole advantage of training horses almost without human supervision. However, as each horse runs in a partitioned space, the actual racing conditions and the impact thereof on the physiology of the horse and on its performance at each moment cannot be revealed. Thus this device does not make it possible to monitor the appearance of a defect in the movements of a horse, for example when it is running in a straight line or on a bend, or in a fatigue situation.

In the field of the training and monitoring of racehorses, a more sophisticated system intended for the instrumentation of racehorses, in the context of use in an actual racing or training situation, is also known from the document US 2007/0130893. This known system is based on the fixing of several movement sensors distributed over the body of the horse, and in particular on its legs or on its hooves. Each sensor is located on and is associated with a part of the body of the animal. The various sensors distributed are next connected by a wireless link through a router also placed on the horse.

This system has several drawbacks.

As a matter of fact, it is complex because of a multitude of components that must function together and communicate with each other in real time. In addition, in so far as it exists in reality, it would be very difficult to implement in practice. A racehorse, in particular a thoroughbred intended for flat racing, is extremely nervous and timorous, or even highly strung, and making it be patient to equip it with the various components required by the system described can scarcely be considered. For example, it appears scarcely conceivable to place sensors on the hooves of such a horse, or to remove them. This difficulty would be further increased if it were a case of equipping several horses in particular in order to compare their performances during training or a race.

Assuming nevertheless that this obstacle could be overcome, this document also poses the major problem of the quality and processing of the signals received. Sensors placed on the limbs of a racehorse will modify its running, so that the signals received will be difficult to interpret, contrary to the objective of precision and reproducibility that is sought.

In addition, a sensor fixed to the leg of a horse, for example by means of a shin boot if the horse is a steeplechaser, is liable to have a position that is upset according to at least two degrees of freedom, namely the height on the leg and the degree of rotation with respect to the initial fixing. For four legs, it would be necessary to manage eight degrees of freedom, which will give rise to an accumulation of margins of error in positioning the sensors, which will have a negative effect on the precision of the calculations. In addition, the digital processing operations for taking account of the stray movements of the sensors will be very heavy, which will make it impossible to obtain the required results, or significant extra expense. These drawbacks would be even further prohibitive in the case where several horses were to be monitored simultaneously.

Moreover, in other fields of application, products exist on the market specially designed for monitoring the physiological parameters of sportspersons, but they also have drawbacks. In particular, the acquisition systems thereof do not include a heart-frequency meter and a precise movement sensor in a single measuring box, and they are incapable of drawing the complete curve of the cardiac signal, and the essential parameters thereof. In addition, they cannot be used as peripherals of portable computer equipment since they use highly specific communication protocols with their data processing base.

Finally, the known products are not adapted for use with a plurality of carriers, as may be necessary for example in order to monitor the physiological parameters of members of a sports team simultaneously.

### AIMS OF THE INVENTION

In the particular case of racehorses, one aim of the invention is to provide a simple system easy to implement, suitable for solving the drawbacks of the known systems of the prior art.

Another aim of the invention is to provide a system that can indicate the change in certain physiological parameters of a horse or a group of horses, according to the evolution thereof on the racecourse, with in particular the objective of distinguishing certain muscular, articular or other dysfunctions, according to the fatigue of each horse, and according to its position on the racecourse.

Generally speaking, one aim of the invention is consequently to provide a reliable system for acquiring and processing data representing the physical activity or the physiological state of a human or animal individual, or a group of such individuals. This system must make it possible to deliver, in real time, on the one hand a signal representing the spatial movements of each individual and, to deliver on the other hand a signal representing a physiological value of each individual, while ensuring that the movement signals relating to each individual are of high precision and are synchronised with the respective physiological signals in order to be able not only to study each instantaneous signal as such but in particular to study the evolution of each signal synchronously, and consequently in correlation with the physical activity of each individual. The aim sought is in fact for the system to make it possible to indicate how such and such physiological parameters of the individual evolve according to its situation, in particular according to its travel on the ground.

Another aim of the invention is to provide a system for storing the acquired data representing the physical activity of the human or animal individual, locally and/or remotely from the individual, in order to be able to analyse the acquired data in real or deferred time.

Another aim of the invention is to provide a system for reliably and quickly transmitting the acquired data representing the physical activity of the individual over a distance which can selectively be close, medium or long.

Another aim of the invention is to provide a system easily adaptable to a variety of situations, ranging from the monitoring of racehorses on a racecourse, to the monitoring of an individual sportsperson or a plurality of sportspersons on a sports field, or monitoring an individual in particular work situations.

Another aim of the invention is to provide a system that makes it possible to simultaneously monitor the performances of several individuals, while also offering the possibility of varying the level of detail of the individual monitoring, in real time.

In the field of the monitoring of racehorses or animals, one principle of the invention on the contrary consists (unlike the teachings of the document US 2007/0130893, which provides to place sensors as close as possible to each movement to be analysed, i.e. on the limbs of the horse) in centralizing several movement sensors and several physiological sensors in a single electronic box for each horse, this box being intended to be positioned at a point on the body of the animal remote from the limbs and preferably close to the animal's centre of gravity, where said single box will not generate any impact on the animal's movements.

Reliable and reproducible movement signals will be thus be obtained, from which quality information on the movements of the animal will be extracted, by means of appropriate processing algorithms applied to the values measured by the sensors in the single electronic box.

In order to further improve the result of the algorithmic processing operations performed on the data sent by the sensors, it is useful to correct the positioning of the sensors used, where the positioning of the sensor box slightly changes during the race or training of the horse.

As a matter of fact the positioning of the electronic box containing the movement sensors is necessarily subject to errors due to the actual constraints. It is therefore important to correct this error before any use of the data for the purpose of analysis or measurement. The fact that all the sensors are located within a single non-deformable box makes it possible to know that the error would be the same for all the sensors, which is not the case in the measurement systems based on several measurement boxes not mechanically integral with each other, or even completely independent as described in US 2007/0130893.

Such a positioning error corresponds to a change in the basic reference frame with respect to the perfect theoretical positioning, so that it is possible to correct it by applying the reverse base change. This base change operation is performed after having calculated the base change matrix by analysing the correlations between the series of measurements sent by the various sensors (accelerometer, gyroscope, etc), with the reference series being decorrelated.

Generally, the subject matter of the invention is a system for acquiring and processing data representing the physical activity of a plurality of human or animal individuals, characterised in that it comprises, for each individual, a single individual electronic box containing several sensors able to measure physical and/or chemical quantities relating to the physical activity of the carrier of the box, each electronic box also being provided with means of radio communication with a remote device for managing the data collected from the individual boxes.

Preferably, each individual electronic box contains movement sensors adapted to deliver in real time a signal representing the spatial movements of the individual, and at least one physiological sensor able to deliver a signal representing at least one physiological quantity of the individual, as well as a computer receiving as an input the signals from the sensors and delivering as an output synchronised and digitised signals, and a memory for storing said synchronised and digitised signals.

The means of synchronising the movement signals and the physiological signals comprise a computer receiving the signals from the sensors as an input and delivering synchronised signals as an output, either to a local memory situated in a local receiver or to a remote memory situated in a remote receiver, by adapted transmission means.

Thanks to this structure, the system according to the invention makes it possible to couple the physiological data with the corresponding movements of the individual.

In particular, in the case of a racehorse, it will be possible to monitor the evolution, for example, of a state of muscular fatigue according to the actual position of the horse on a racecourse and the effort already made, and thus detect muscular fatigue, or the appearance of abnormalities in the movement, while being able in particular to separate the race phases in a straight line and on a bend.

Equipping several horses simultaneously with an individual electronic box and synchronising them with a remote management device will make it possible to have a kind of team sensor, corresponding to all the individual sensor boxes, and making it possible to simultaneously acquire and process the physiological parameters of several horses or more generally of several individuals.

Preferably, communication between the individual electronic boxes and the remote management device is bidirectional and also makes it possible to send information or instructions to each individual electronic box.

In addition, provision can be made for the information and/or instructions to be reproduced by the individual electronic box in the form of audible or voice messages, but other reproduction means are foreseeable: by visual mode, by the generation of electrical pulses, mechanical vibrations, or others.

These features are particularly advantageous in the case of the monitoring of collective sports. All the sensors of the players in a team then constitute a form of virtual team sensor. As a matter of fact the movement and position information or the physiological information of each player are received in real time by the trainer's display and management device who can then have a global perception of the situation and take decisions or give individual instructions accordingly.

Advantageously, the remote management device is provided with means for adjusting in real time the bandwidth of the radio communication with each individual electronic box. It will thus be possible for the user of the remote management device, typically the trainer, to zoom in on or to magnify the physical activity of such and such player in the team.

As these magnifying effects will require more instantaneous bandwidth for the communication between the remote management device and the individual box concerned, it will be useful to be able to vary, from the remote management device, the bandwidth allocated to the communication with each individual electronic box.

In one conceivable mode, the radio communication between the individual electronic boxes and the remote management device is achieved using a mobile telephony communication network, in particular of the GSM type.

Alternatively, the radio communication between the individual electronic boxes and the remote management device is done by means of a dedicated autonomous communication network, in particular of the Zigbee type.

When monitoring the positions of the individuals on the ground is necessary, the system according to the invention also comprises positioning beacons distributed around the area of activity of the individuals, with respect to which each electronic box determines its instantaneous position and transmits it to the remote management device.

Provision can be made for the positioning beacons to comprise a communication relay function between the individual electronic boxes and the remote management device, so as to ensure continuity of radio communication between the individual electronic boxes and the remote management device when the latter is beyond the direct range of communication of the individual electronic boxes.

Ideally, the individual electronic boxes and the remote management device are configured to function as an auto-router and auto-adaptive radio communication network able to automatically optimise the communication path between the individual electronic boxes and the remote management device, according to the movements of the individuals.

According to the nature of the individual and his/her physical activity, it may be useful to use on site all or some of the movement signals and physiological signals captured, but it may also be useful to use these signals at a distance, whether in real time or deferred time. Consequently the system according to the invention comprises means for transmitting the movement signal or physiological signal to a local or remote storage unit for storing and processing the signals received.

According to the architecture adopted for the system in its application context, the data sent by the sensors to the local receiver and/or the remote receiver can be transmitted in several ways.

According to a first embodiment, the transmission means are coupled to the local receiver by means of a short-range wireless link, typically of around a few metres, and this wireless link is then in particular of the Bluetooth or Zigbee type.

According to another embodiment, the transmission means are coupled to the remote receiver by means of a medium-range wireless link, typically of around a few hundreds of metres, and for this purpose a connection of the Zigbee type may also be suitable. The data transmitted may optionally be relayed by Zigbee routers, which extends the range of the network.

According to another embodiment using a longer-distance transmission between the local receiver and a remote receiver associated with a centralized server, the local receiver is coupled to the remote receiver by means of a long-range wireless link, in particular by means of a wireless mobile telephony network of the GSM type or equivalent.

Advantageously, the computer is associated with display means for displaying data sent by the movement sensors and physiological sensors, and/or graphical representations of these data.

The system according to the invention affords great flexibility according to the actual activities to be analysed. This flexibility is in particular based on the variety of movement sensors and physiological sensors that can be used.

Advantageously, the movement sensor is taken from a set of sensors comprising an accelerometer, a gyroscope, a magnetometer or a pressure sensor.

Alternatively or in addition, the movement sensor is taken from a set of relative positioning beacons, including beacons of the GPS type (standing for Global Positioning System), ultrasonic beacons, beacons based on an ultra wideband network, or beacons based on a communication protocol of the Zigbee type.

According to the invention, the physiological sensor is taken from a set of sensors comprising ECG-EKG cardiac signal sensors, EMG muscular activity sensors, EEG cerebral activity sensors, body temperature sensors, blood circulation sensors and embedded blood analysis sensors.

In the case of the monitoring of racehorses, the system is preferably configured so that the movement sensors and the physiological sensors are coupled by means of a short-range transmission, in particular of the Bluetooth or Zigbee type, to a local receiver in the form of a terminal of the personal digital assistant type or mobile telephone type. Thus the jockey can, in real time during the race and/or training, monitor certain physiological parameters of the horse, or even adapt the pace of the horse accordingly.

In a variant, the movement sensor and physiological sensor boxes are coupled by a medium range transmission, of around 300 metres, in particular of the Zigbee type, to a remote terminal, connected to display processing means 23, for example a personal computer. This remote reception terminal is in particular used by a trainer of the sportsperson or team of sportspersons being monitored, and it has means of bidirectional communication with the individual sensor boxes, so as to be able to transmit information or instructions thereto.

In another variant, the movement sensors and physiological sensors are coupled, by a transmission of any range, in particular of the GSM type, to a remote server.

The movement sensors used in the case of the monitoring of a racehorse comprise for example a triaxial accelerometer, a GPS sensor for providing the movement path of the horse, and the physiological sensors comprise an electrocardiograph supplying the cardiac signal of the horse in real time. Optionally, the physiological sensors can also comprise in particular a blood analysis sensor. The sensor box is fixed by a strap under the belly of the horse. In addition, all the movement sensors are located within a single non-deformable box so that any change in the positioning of said box will result in an equivalent change for all the movement sensors, corresponding to a change in the reference base able to be corrected by applying a reverse base change.

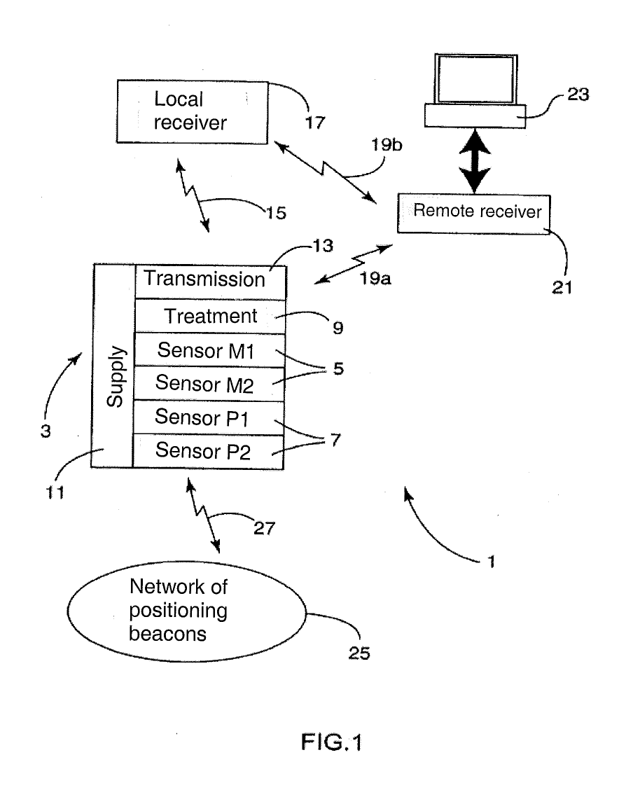

The features and advantages of the invention will emerge from a reading of the detailed description of the accompanying drawings, in which FIG. 1 illustrates a general functional flow diagram of the system according to the invention.

Reference is made to FIG. 1. In this figure, a functional flow diagram of the system 1 according to the invention has been shown.

This system 1 comprises firstly, for each individual whose movement parameters and certain physiological parameters it is wished to monitor, an electronic box 3 for acquiring the movement signals and physiological signals. This box 3 comprises at least one movement sensor 5 and at least one physiological sensor 7. These sensors are grouped together within the same box which, combined with a suitable location of the box on the individual, minimises errors on the captured signals. They are connected to a local pre-processing unit 9, either by a cable link or by a wireless link, typically short range, in which case the sensors are auto-supplied. If not so, the sensors can be supplied by a common supply 11 forming an integral part of the acquisition box 3 and also supplying the unit 9 and a wireless transmission unit 13. The outputs of the sensors are connected to the wireless transmission unit 13, which transmits the signals sent by the sensors at a distance.

According to the nature of the sensors, which will be detailed below, it may be useful to perform, in the processing unit 9, a local pre-processing of the analogue or digital signals sent by the sensors 5, 7 in order to locally obtain, on the acquisition box 3, certain parameters sent by the sensors. In this case it is rather the output of the processing unit 9 that is connected to the transmission unit 13, instead of the output of the sensors 5, 7.

The transmission unit 13 can be connected by a short-range wireless link 15, in particular of the Bluetooth or Zigbee type, to a local receiver 17, which in particular receives and displays locally the movement parameters and the physiological parameters sent by the sensors, or the processing unit 9 where it is useful to have movement information and physiological information already somewhat synthesised in order to be understandable to the user of the system.

Thus, in an advantageous embodiment of the invention, the local receiver 17 will be formed by a mobile communication box, of the PDA (Personal Digital Assistant) type or of the mobile telephone type, having in particular capabilities for the real-time display of information sent by the sensors, and in particular storage and processing capabilities. This configuration is in particular useful when monitoring racehorses since it enables the jockey to display the physiological parameters of his/her horse in real time on a local receiver.

According to the invention, it may also be useful to transmit the data sent by the sensors at a greater distance in order to store them and process them in greater details, whether in real time or deferred time.

For this purpose, the invention provides to connect the transmission unit 13 of the sensor box 3 to a remote receiver 21 by means of a medium-range wireless link 19a.

In a variant or in addition, it is possible, according to the requirements of the specific application, to provide for the connection of the local receiver 17 to said remote receiver 21, by means of a medium- or long-range wireless link 19b.

Thus, in the particular case of the monitoring of racehorses, the jockey will be able to have available information at the heart of the action, by means of his/her local receiver 17, but the stable will be able to have the same information, or even more complete information, at the edge of the racecourse, by means of the remote receiver 21 connected by a wireless link either to the sensor box 3 or to the local receiver 17.

Naturally, in order to completely use the movement data and the physiological data sent by the individual wearing the sensor box 3, the remote receiver 21 is preferably connected to a processing and display station 23, which can for example consist of a personal computer.

It should be noted that the physiological quantities that it is sought to capture and analyse are directly sampled by the physiological sensors 7. With regard to the movement sensors 5, these are situated solely on the sensor box 3, which is self-contained in its task of acquiring movement quantities and physiological quantities. However, it may also be necessary to monitor the spatial movements of the individual with reference to a network 25 of absolute-positioning beacons, external with respect to the individual, such as for example the network of positioning beacons of the GPS (Global Positioning System) type, or other. In this case, the transmission unit 13 of the sensor box 3 will also be connected by a wireless link 27 to the network of positioning beacons 25.

The electronic acquisition boxes 3 and the sensors that they use will now be described in greater details.

Each individual electronic box performs precise measurements on an individual. These measurements are as follows:
Position measurements, which are performed by means of an absolute positioning system (for example of the GPS type) or relative positioning system (for example by triangulation of the position with respect to beacons situated at fixed locations).
Movement measurements, which are performed by means of a triaxial accelerometer, and/or a triaxial gyroscope. They take account of the spatial movements of the individual wearing them.

Cardiac measurements, which are performed by means of an EKG sensor and capture the cardiac signal and deduce therefrom certain characteristic parameters, such as for example the heart rate (for example to detect any abnormalities) of the sportsperson at any time.

All these sensors are known per se and consequently will not be the subject of a detailed description.

The box 3 also transmits the information by a short-, medium- or long-distance wireless link (which may be greater than 1 km). Each box 3 can be connected to a receiver particular to it or to a common receiver. In the first case (one receiver per box), the data coming from each of the boxes are then centralized to the computerised processing, display and storage system, such as the remote receiver 21 associated with the station 23. In the second case (a common receiver for all the boxes), the data are transmitted to a single reception box capable of identifying the origin of the messages received and to transmit them to the computerised processing, display and storage system. This computerised storage system:
displays, in real time on an interactive monitoring screen, the position of the individuals (horses, sportspersons, etc) in their environment, and the individual and collective information relating to them;
records the data for subsequent use, in particular subsequent analysis and long-term monitoring of the individuals;
analyses, in real time or subsequently, the individual or collective information on the individuals equipped, according to previously defined criteria.

Deferred-time analysis of the performance by means of the system according to the invention has already been experienced by the applicant and perfectly operates in the equine field. It can easily be adapted for improving the monitoring of the physical activity of other types of individual and for other individual or collective disciplines.

Examples of movement sensors and physiological sensors capable of being used for implementing the invention will now be given:

The sensors present in the system can be divided into two classes: movement sensors and physiological sensors.

Among the movement sensors, there are:
The accelerometer: known in the prior art, this makes it possible to know the acceleration in n directions (with n=1, 2 or 3).
The gyroscope: known from the prior art, this makes it possible to know the speed of rotation of a body according to n rotation axes.

The accelerometer does not make it possible to determine the position and speed, which are the integrals of acceleration. The gyroscope does not make it possible to determine the real angles, which are the integrals of the rotation speeds. In either case, it is necessary to determine the integration constants. For this purpose other movement sensors are used, from the following:
The magnetometer: known from the prior art, this makes it possible to obtain an angle with respect to the direction of a locally present (possibly terrestrial) magnetic field.
The pressure sensor: known from the prior art, this makes it possible to measure the variation in altitude and depth considering the constant ambient pressure at a fixed altitude.

It is also possible to monitor the spatial movements of an individual by means of a beacon positioning system, among which it will be possible to provide several possibilities:
GPS beacons: known in the prior art, these make it possible to obtain the position of the sensor (and therefore its speed by derivation) with respect to the terrestrial reference frame. They are relatively imprecise (precision of approximately 10 m in normal mode and 2 m in differential mode) and have a low acquisition frequency (1-5 Hz).
Ultrasonic beacons: these are known from the prior art and afford precise positioning (1 cm) in a small environment (a few hundred metres) where ultrasonic transmitters are placed.
Ultra wideband beacons: these are known from the prior art, and afford fairly precise positioning (10 cm) in a small environment (a few hundred metres) where transmitters are placed.
So-called Zigbee beacons: these are little known in the prior art and afford fairly imprecise positioning but using as a beacon a communication network using a Zigbee communication protocol also used for data transmission.

Beacon systems make it possible to know (with precision depending on the system chosen) the position with a frequency dependent on the system chosen but relatively low (around a few dozens of Hz at a maximum).

In order to improve further the monitoring of movements, the invention provides to couple certain movement sensors within the acquisition box 3. The following will be mentioned:
Coupling an accelerometer and a beacon position sensor: this makes it possible to obtain precise information on the position with a high sampling frequency (1 kHz or more). The accelerometric data are integrated once or twice (to obtain the speed or position), and drifts due to an offset on the sensor or a wrong integration constant are corrected by means of the positioning system (which gives an absolute position or speed). This will be referred to hereinafter as a compensated accelerometer.
Coupling an accelerometer and a pressure sensor: this makes it possible to obtain precise information on the height or depth with a high sampling frequency (1 kHz or more). The accelerometric data are integrated twice and drifts are corrected by means of the pressure sensor (which gives the absolute altitude). This will be referred to hereinafter as a compensated altimeter.
Coupling a gyroscope and a magnetometer: this makes it possible to obtain very precise information (<1° error) and high frequency (1 kHz) on the angle of the sensor. The principle is the same as before: the gyroscope data are integrated once and any drift is corrected by means of a magnetometer. This will be referred to hereinafter as a compensated gyroscope.

According to the invention, a physiological sensor is associated with one of the movement sensors described above so as to be able to precisely associate the values representing the spatial movement of the individual with some of the values describing its corresponding physiological activity.

Among physiological sensors, the following will be mentioned:
The electrocardiograph (ECG or EKG): known in the prior art, it makes it possible to obtain the ECG signal relating to the heartbeat. It may have several channels, and each channel uses an electrode, to which it is necessary to add a common earth electrode for all the channels. The acquisition is effected at high frequency (>500 Hz) so as to be able to finely observe the signal.
Electromyography (EMG): known in the prior art, this analyses the electrical activity of the muscles. Acquisitions also take place at high frequency (>500 Hz).
Electroencephalography (EEG): known in the prior art, this analyses the electrical activity of the brain. The acquisitions also take place at high frequency (>500 Hz).
Temperature sensors: well known in the prior art, these measure temperature. Provision is made for using several of these for measuring temperature at various points on the body of the individual during a test. The general architecture of the acquisition or processing system 1 as just described may be suitable for monitoring the activity of individuals in a large number of application situations, by means of a few adaptations within the capability of a person skilled in the art according to each specific application.

Some specific applications and the corresponding adaptations of the system 1 will now be described.

Thus the movement sensors and the physiological sensors previously described can be suitably coupled with each other according to the type of test performed, several examples of which will now be given.

The locometry test: this type of test aims at determining the individual mechanical characteristics during an effort in real situation. This test is particularly useful, in the context of the invention, to the monitoring and analysis of the movement parameters and physiological parameters in the case of racehorses. It requires the use of a team sensor composed of one or more movement sensors, compensated or not. The sensors communicate with a computerised data display and recording terminal for analysing in real or deferred time and in a comparative manner the performances between several horses that are being tested or the performances of which have already been previously recorded. The team sensor also enables the supervisor to focus on one individual in particular so as to refine the diagnosis.

The movement sensors record at high frequency the data relating to this movement so as to reveal, by means of signal processing methods and algorithms (not described here) the characteristics of the movement, and possibly the problems that are related thereto.

In the case of the application to a horse, each movement sensor comprises a triaxial accelerometer, a GPS sensor providing the tracked travel of the horse, and an electrocardiograph providing the cardiac signal of the horse in real time. The use of a physiological sensor of the EMG type coupled to the previous one make it possible to detect, in addition to the mechanical characteristics and problems of the subject, its muscular characteristics and problems.

In addition, the use of compensated movement sensors may be of great interest for refining the measurements and makes it possible in fact to know more precisely the movement to a timescale of several seconds. This information makes it possible to correlate the high-frequency measurements characteristic of the locomotive functioning of the subject in overall movement. This makes it possible to reveal phenomena such as a locomotive problem during a phase where the subject is moving with a characteristic movement (for example in a curve, on a rise or on a descent etc).

Effort, Endurance or Movement Tests

This type of test aims at determining the potential of an individual in the context of a given effort test.

In general a movement sensor using a compensated accelerometer and optionally a gyroscope (optionally compensated), coupled to physiological sensors, are used. Apart from the locometric “micro” data recorded at high frequency and described previously, the advantage of these tests is to generate more synthetic “macro” data at a lower frequency (typically 1 to 5 Hz, which limits the bandwidth necessary for wireless transmission) making it possible to characterise the evolution of certain physiological and locometric parameters during a test. Thus, by way of example, in the field of cardiac signals, a micro-data item will consist of the whole cardiac signal as a function of time, and a macro-data item sent by the micro-data item will consist of the heart rate in beats per minute.

The sensors used are those of locometric tests, except that it is essential to use compensated sensors so as to know data such as the speed or the path followed by the subject rather than only his acceleration or instantaneous rotation speed. It is possible to add other sensors: one or more temperature sensors for obtaining at regular intervals (with a frequency of approximately 1 Hz), additional information on the body temperature of the subject under particular external temperature conditions, an electroencephalogram EEG for recording the evolution of brain activity during a test (for example an endurance test).

The matching, in particular by the processing unit 9, of the ECG, EMG, EEG “macro” data and data describing the movement of the individual makes it possible to obtain particularly relevant coupled information such as: muscular and cardiac activity corresponding to a given isolated effort during an effort test, recovery time after effort (recovery test), change in heart rate and muscular and brain activity and characteristics of movement during a long-duration test (endurance test) or in the context of an activity involving several subjects (collective sport etc).

Advanced Physiological Tests, Such as Measurement of Stress

This type of measurement aims at characterising the behavior of a subject in a stress situation. It requires the use of a sensor of the ECG and/or EEG type optionally coupled with a movement sensor (non-compensated). This assembly makes it possible to observe the evolution of the heart rate and movements due to tension and stress when a subject is subjected to a stress test. For this purpose the system according to the invention is adapted and comprises at least one movement sensor from among a triaxial accelerometer, a magnetometer, a pressure sensor, a gyroscope and a position sensor of the GPS type, ultrasonic sensor of the Zigbee or ultra wideband type, and at least one physiological sensor taken from among an electromyography, at least one temperature sensor, an electrocardiograph ECG, and an electroencephalograph EEG. Preferably each of said sensors has at least one channel.

The physiological tests thus conducted by means of the aforementioned sensor boxes are particularly adapted to the management of stress in a set of jobs at risk, in particular firefighters or police officers.

The Monitoring of Sportspersons Moving in a Delimited Space Such as a Sports Hall or Ground

The system according to the invention can also be adapted to this type of application.

The system is used as a team sensor in that the supervisor (trainer or other) can display, record and replay on a computer terminal the data relating to his/her players: position on the ground, state of fatigue, distance traveled, physical characteristics at the time of measurement, history of physical characteristics, for example.

The supervisor can also choose at any time to refine the measurement on a particular individual so as to be able to carry out a fine analysis, or even a real-time medical diagnosis on him/her. In this case, the team sensor automatically adapts its data flows so as to get more information from the individual concerned.

The movement sensors comprise at least three ultrasonic beacons coupled to a radio synchronisation device, and each player is provided with an ultrasonic sensor for measuring as required: the distances between each beacon and the sensor (the sensor then requires radio synchronisation with the transmitters) or the differences between these distances (the sensor then does not require synchronisation with the transmitters but the processing operations to be performed are more complex), so as to determine the relative position of each player with respect to all the transmitters with a frequency of around 5 to 10 Hz.

In the case where the sportspersons are moving out of doors, the system must be adapted and the movement sensors comprise beacons of the GPS type, optionally coupled to ultrasonic beacons, Zigbee beacons or ultra wideband beacons.

In both cases, the physiological sensors comprise a heart frequency meter, the signal of which is processed so as to obtain each player's heart rate.

Supply of Data to Remote Applications of the Web Type

The system according to the invention can also be adapted to this type of application. In this case, the data from the team sensor consisting of a set of acquisition boxes and a supervision and recording unit are transmitted to a remote server using a link of the GSM type (mobile telephone). Then they are stored in a data base so as to be used by specific web applications, such as for example social networks on the internet, or equivalent. The remote web server processes the data according to specific application algorithms, which are not described here, but which are within the capability of a person skilled in the art.

### ADVANTAGES OF THE INVENTION

The advantage of the system according to the invention is to provide a sensor platform that is flexible and easily adaptable to several types of application, such as the monitoring of the performance of racehorses, or monitoring human activity, sports or otherwise. The system includes in particular in a single electronic box a movement sensor and a heart frequency meter, and can ultimately include several additional sensors if required.

In some applications, such as the monitoring of the movement parameters of racehorses, fixing and locating the single electronic box under the belly of the horse, as provided for by the invention, is particularly important for the final results, and considerably simplifies the processing of the signals, in particular those relating to the movements of the horse, in order to extract therefrom information on the existence and location of such and such problem, for example with regard to an articulation of a leg.

The sensor box functions as a mobile telephone peripheral, PDA or standard computer terminal, which avoids the purchase of a dedicated receiver.

The use of the system makes it possible to provide and analyse movement data and corresponding physiological data, with a much greater level of precision and detail than in the past, which in particular optimises the training of racehorses, or sportspersons practicing a collective or individual sport.

In particular, in the case of racehorses, it will be possible to monitor the evolution, for example, of a state of muscular fatigue according to the actual position of the horse on a racecourse and the effort already made, in comparison with other horses making a similar effort simultaneously, and thus to detect muscular fatigue, or the appearance of abnormalities in the movement, while being able in particular to separate the phases of racing in a straight line and on a bend.

The acquisition of data takes place in wireless mode on a terminal of the mobile telephone type, PDA, PC notebook or a computer equipped with a radio link (for example Bluetooth or Zigbee) so as not to interfere with the movements of the animal or individual.

The strong points of the system are mainly the recovery of precise and complete information on the performance of individuals (biomechanical, cardiology and movements), the display, comparison and analysis in real time of the evolution of this information individually and collectively, whether locally or at a distance, and the possibility of long-term monitoring of individuals, making it, possible to optimise their collective and individual performances.

The possibility of automatically and dynamically adjusting between a sensor box and the remote management device makes it possible to zoom in on the physical activity of an individual in a team.

The invention also makes it possible to carry out an advanced technical assessment of the biomechanical and physiological performance of humans or animals.

1. A system for the acquisition and processing of data representing the physical or mental activity and/or the physiological state of a plurality of human or animal individuals, comprising, for each individual, a single individual electronic box containing several sensors able to measure physical and/or biological values relating to the physical activity of the carrier of the box, each electronic box also being provided with means of radio communication with a remote management device for managing the data collected from the individual boxes.

2. A system according to claim 1, wherein each individual electronic box contains movement sensors able to deliver in real time a signal representing the spatial movements of the individual, and at least one physiological sensor able to deliver a signal representing at least one physiological value of the individual, as well as a computer receiving the sensor signals as an input and delivering synchronised and digitised signals as an output, and a memory for storing said synchronised and digitised signals.

3. A system according to claim 1, wherein the communication between the individual electronic boxes and the remote management device is bidirectional and also makes it possible to send information or instructions to each individual electronic box.

4. A system according to claim 3, wherein the information and/or instructions received are reproduced by the individual electronic box in the form of voice messages or light, audible or electrical signals.

5. A system according to claim 3, wherein the remote management device is provided with means for adjusting in real time the bandwidth of the radio communication with each individual electronic box.

6. A system according to claim 3, wherein the radio communication between the individual electronic boxes and the remote management device takes place by means of a mobile telephony communication network.

7. A system according to claim 3, wherein the radio communication between the individual electronic boxes and the remote management device takes place by means of a dedicated self-contained communication network.

8. A system according to claim 7, further comprising positioning beacons distributed around the area of activity of the individuals, with respect to which each electronic box determines its instantaneous position and transmits it to the remote management device.

9. A system according to claim 8, wherein the positioning beacons comprise a function of communication relay between the individual electronic boxes and the remote management device, so as to ensure continuity of the radio communication between the individual electronic boxes and the remote management device, when the latter is beyond the direct range of communication of the individual electronic boxes.

10. A system according to claim 7, wherein the individual electronic boxes and the remote management device are configured so as to function as an auto-routing and auto-adaptive radio communication network, able to automatically optimise the communication path between the individual electronic boxes and the remote management device.

11. A system according to claim 2, wherein at least one movement sensor is taken from a set of sensors including an accelerometer, a gyroscope, a magnetometer, a pressure sensor, a compensated accelerometer, a compensated altimeter and a compensated gyroscope.

12. A system according to claim 2, wherein at least one movement sensor is taken from a set of positioning beacons, including beacons of the GPS type, ultrasonic beacons, beacons based on an ultra wideband network, and beacons based on a communication protocol of the Zigbee type.

13. A system according to claim 2, wherein said at least one physiological sensor is taken from a set of sensors, including ECG-EKG (electrocardiograph) cardiac signal sensors, EMG (electromyography) muscular activity sensors, EEG (electroencephalograph) brain activity sensors, body temperature sensors, blood pressure sensors, and embedded blood analysis sensors.

14. A system according to claim 1, wherein it is optimised for monitoring the physical activity of one or more horses on a racetrack or an equestrian competition area, and it comprises, for each horse, a single individual electronic box, fixed away from the limbs and in the vicinity of its centre of gravity and containing several sensors able to measure physical and/or biological values relating to the physical activity of each horse, each electronic box also being provided with means of radio communication with a remote device for the management of data collected from each individual electronic box.

15. A system according to claim 14, wherein said individual electronic box is fixed by a strap under the belly of the horse.

16. A system according to claim 14, wherein the sensors include movement sensors that comprise a triaxial accelerometer, a GPS sensor providing the tracked travel of the horse, and physiological sensors that comprise an electrocardiograph supplying the cardiac signal of the horse in real time.

17. A system according to claim 14, wherein the sensors include a blood analysis sensor.

18. A system according to claim 15, wherein all movement sensors are located within a single non-deformable box so that any drift in positioning of said box will cause an equivalent drift for all the movement sensors, corresponding to a change in reference base and able to be corrected by applying a reverse change in base.

19. A system according to claim 1, wherein it is optimised for monitoring the physical activity of one or more players moving on a sports field, and it comprises, for each player, a single individual electronic box containing several sensors able to measure physical and/or biological quantities relating to the physical activity of each player, each electronic box also being provided with means of radio communication with a remote management device for managing the data collected from each individual electronic box and managing the network of sensor boxes.

20. A system according to claim 19, optimised for monitoring sportspersons moving in a delimited place such as a sports field or hall, wherein movement sensors positioned in the individual electronic box comprise at least three ultrasonic beacons coupled to a radio synchronisation device, and each player is provided with an ultrasonic sensor for measuring as required: the distances between each beacon and the sensor or the differences between these distances, so as to determine the relative position of each player with respect to all the transmitters with a frequency of around 5 to 10 Hz.

21. A system according to claim 19, optimised for monitoring sportspersons moving outdoors, wherein movement sensors positioned in the individual electronic box comprise beacons of the GPS type.

22. A system according to claim 19, wherein the physiological sensors positioned in the individual electronic box comprise a heart frequency meter the signal of which is processed so as to obtain the heart rate of each individual.

23. A system according to claim 1, optimised for providing data to remote applications of the web type, adapted to transmit the data from the sensors to a remote server using a telephone link, said remote web server executing specific web applications.

24. A system according to claim 1, optimised for detecting the stress in an individual in a work situation, wherein movement sensors positioned in the individual electronic box comprise an accelerometer and physiological sensors comprise a heart frequency meter and an electroencephalograph.

25. A system according to claim 1, optimised for advanced physiological tests on individuals, wherein movement sensors positioned in the individual electronic box comprise one or more sensors taken from a triaxial accelerometer, a magnetometer, a pressure sensor, a gyroscope and a position sensor of the GPS type, and an ultrasonic sensor of the Zigbee or ultra wideband type, and physiological sensors comprise one or more sensors taken from an electromyograph, at least one temperature sensor, an electrocardiograph ECG, and an electroencephalograph EEG, each of said sensors having at least one channel.
